(19) United States
(12) Patent Application Publication
(10) Pub. No.: US 2017/0355909 A1
(43) Pub. Date: Dec. 14, 2017

(54) Mobile apparatus for carbon-containing materials including biohazard wastes gasification by thermal decomposition and conversion into a liquid fuels.

(71) Applicant: Alexander Dementev, Etobicoke (CA)

(72) Inventor: Alexander Dementev, Etobicoke (CA)

(21) Appl. No.: 15/180,061

(22) Filed: Jun. 12, 2016

(51) Int. Cl. C10G 2/00; B09B 3/00; C25B 1/02; C10J 3/20; A61L 2/07; C10J 3/06

(52) U.S. Cl. CPC C10G 2/30; A61L 2/07; C10J 3/06; C25B 1/02; C10J 3/20; B09B 3/0091; C10J 2300/1659; C10J 2300/0946; C10G 2300/4068; C10J 2200/31

(57) ABSTRACT

The present invention relates to a method for gasification of carbon-containing materials including biohazard wastes, and more specifically, to a method for gasification of carbon-containing materials which allows an increase in carbon efficiency and a reduction in carbon dioxide emission, comprising the steps of: biohazard wastes grinding and sterilization, mix with carbon-containing materials for the gasification; and catalytic production of diesel fuel. A system having a movable platform including: material preparation block, gasification and catalytic of diesel fuel production reactors which are structurally and functionally integrated. In the practice of the process, a mixture of carbon-containing materials, a compressed air feed and process steam is fed to the gasifier to produce a synthesis gas. The synthesis gas is fed to the Fischer-Tropsch reactor where it is catalytically reacted to produce heavy hydrocarbons. The outlet from the Fischer-Tropsch reactor is separated into water, a low heating value tail gas, and the desired hydrocarbon liquid product. The water is pressurized and heated to generate process steam. The system further includes a plurality of heat exchangers that enable heat to be recovered from the outlet of the gasifier. The recovered heat is used to make the process steam as well as to preheat the hydrocarbon mix before it is fed to the gasifier and preheat the synthesis gas before it is fed to the Fischer-Tropsch reactor. The method of the present invention greatly increases carbon efficiency and reduces the generation of carbon dioxide.

### BACKGROUND OF THE INVENTION

### 1. Technical Field

The present invention relates generally to a system and process for converting carbon-contained materials to a synthetic hydrocarbon liquid and, more particularly, to carbon-contained materials conversion system utilizing municipal solid and biohazardous wastes, coal, used machine oil and tires, plastic, wet wastes and biomass and process employing a material preparation system, gasifier and a Fischer-Tropsch reactor constructed on a movable platform. The system will accommodate the possibility for variation in feedstock co-gasification and production of and ultra clean Fischer-Tropsch synthetic fuels.

### 2. Background Information

Energy is fundamental to economic growth. Economies cannot grow and people cannot raise their standard of living without adequate supplies of affordable energy. The global demand for energy is projected to rise by 56% between 2010 and 2040, with the greatest increase in the developing world′.

Gasification, which can provide cleaner energy and products, is not new. Its origin dates back to the late 1700s when an early form of gasification was used in the UK to create “town gas” from local coal reserves. More modern gasification technologies began to evolve prior to and during World War II as Germany needed to create its own transportation fuels after being cut off from oil supplies.

Gasification is a process that enables the production of a combustible or synthetic gas (e.g., H₂, CO, CO₂, and CH₄) from carbon-based feedstock, referred to as carbonaceous feedstock. The gas can be used to generate electricity or as a basic raw material to produce chemicals and liquid fuels. This process enables the production of a gas that can be used for generation of electricity or as primary building blocks for manufacturers of chemicals and transportation fuels.

As useful feedstocks for the gasification process can be any carbonaceous material, the types of feedstock can range broadly. Useful feedstocks can include, but are not limited to, any waste materials, coal, petroleum coke, heavy oils, biomass and agricultural wastes.

Generally, a gasification process consists of feeding carbon-containing materials into a heated chamber (the gasifier) along with a controlled and limited amount of oxygen and steam. Depending on the origin of the feedstock, the volatiles may include H₂O, H₂, N₂, O₂, CO₂, CO, CH₄, H₂S, NH₃, C₂H₆ and very low levels of unsaturated hydrocarbons such as acetylenes, olefins, aromatics and tars. Once a carbonaceous material is converted to gaseous state, undesirable substances such as sulfur compounds and ash may be removed from the gas.

Fischer-Tropsch processes for converting synthesis gas into higher carbon number hydrocarbons are well known. The Fischer-Tropsch process was developed in early part of the 20^{th} century in Germany. It has been practiced commercially in Germany during World War II and later in South Africa. The hydrocarbon products of a Fischer-Tropsch synthesis generally include a wide range of carbon number, ranging from between about 1 and about 100. The end products which may be recovered from the Fischer-Tropsch synthesis product, following separation, hydro processing or other upgrading, include but are not limited to liquefied petroleum gas (“LPG”), naphtha, middle distillate fuels, e.g. jet and diesel fuels, and lubricant base stocks. Some of these end products, however, are more desirable than others for a variety of reasons, including for example, being marketable at a higher margin.

The desirability of an end product of a Fischer-Tropsch synthesis may also be dependent upon geographic location of the Fischer-Tropsch plant.

A number of carbonaceous sources have been used as raw-materials for producing hydrogen and carbon monoxide containing synthesis gas (hereinafter referred as to syngas) which can be fed into the FT process. Originally, coal was used as the primary raw-material, but lately also natural gas has been taken into use in commercial processes. Even more recently various processes have been developed in which biological materials, such as plant oils, plant waxes and other plant products and plant parts or even oils and waxes of animal origin, are gasified and processed to produce a suitable feed.

The technology and process flow utilizes a combination and variation of carbon-contained materials as feedstock that will be converted to synthesis gas. Parts of the process includes mechanisms and equipment to be incorporated in the process flow to control/remove various acid gases including carbonyl sulfide, hydrogen sulfide, methane, hydrochloric acid, mercury and sulfur oxides, sulfur compounds, and carbon dioxide. The remaining clean stream of syngas comprising of only hydrogen and carbon monoxide will be further converted into ultra clean synthetic transportation fuels. No energy plant, research and development data, or global facility exists, with any intent, or proposal that shows a complete process flow for conversion solutions, utilizing the combination and variation of carbon-contained materials as feedstock for clean synthetic transportation fuels production constructed on a movable platform.

There are number of patents relating to different technologies for the gasification of carbon-contained materials for the production of synthesis gases for use in various applications, including US2527197 A; U.S. Pat. No. 2,879,148 A; US3009795 A; U.S. Pat. No. 3,544,291 A; U.S. Pat. No. 3,904,386 A; U.S. Pat. No. 4,265,868 A; U.S. Pat. No. 4,642,125 A; U.S. Pat. No. 3,976,442 A; WO2011129878 A2 and US5961673 A.

There are number of patents relating to a novel process and apparatus for feedstock preparing prior to the gasification including U.S. Pat. No. 6,170,770 B1; U.S. Pat. No. 4,039,425 A; U.S. Pat. No. 3,826,208 A; US20090007484 A1; CA2185330 A1; U.S. Pat. No. 7,354,550 B2; US20140251923 A1; U.S. Pat. No. 4,342,830 and U.S. Pat. No. 5,941,468.

In recent years, government agencies, industries, and other organizations have had to address various problems relating to the handling and processing of organic waste materials including chemical and biological products. The disposal of medical waste is particularly difficult because of the presence of infectious bacteria, viruses, and other pathogens in the waste. The amount of contagious or infectious waste from medical institutions increases every year. The incineration is most reliable from the viewpoint of sterilization. Since, however, most medical instruments are formed of plastic materials; the plastic materials decompose when incinerated to generate gases containing harmful substances. That is, a secondary contamination problem occurs, and it is required to use a large apparatus for treating the exhaust gases. In the sterilization using a liquid chemical, the chemical itself is harmful to a human body, and handling of the chemical and disposal of the wasted chemical may cause secondary contamination. Safety measures and disposal of liquid waste generate additional costs. Moreover, like the case of sterilization using high-pressure steam, the chemical does not fully reach the interiors of waste materials having the form of a pipe or a tube, and it is therefore difficult to achieve complete sterilization.

Canadians produce more than 660,000 metric tons of dry stabilized organic waste materials each year², and The Canadian Environmental Assessment Agency (CEAA) predicts this output will continue to increase in the foreseeable future. In its raw form, organic waste materials are a pollutant subject to strict federal regulation at the hands of the CEAA, and bio solids are similarly regulated by counterpart state and municipal authorities as well. There is a number of patents relating to different technologies for the biohazardous materials handling and processing including U.S. Pat. No. 5,333,146 A; U.S. Pat. No. 6,360,679; U.S. Pat. No. 6,348,174 B1; U.S. Pat. No. 5,084,250 A and U.S. Pat. No. 6,867,393 B1.

When carbon-containing materials are gasified, “fuel gas” is produced consisting of CO, CO₂, H₂, N₂, H₂O, CH₄, and other light hydrocarbons in varying proportions and amounts. Residual tar and oil materials are also produced as by-products entrained in the pyrolysis gases. These materials are extremely viscous, and condense on piping and other equipment in the gasification system. They may also combine with char produced in the system to form layers of a solid organic residue which are extremely difficult to remove.

The gasifier is comprised of a primary chamber for receiving the waste, a fume transfer vent, and a mixing chamber to accept the pyrolysis gases from the primary chamber. The fumes then flow to an afterburner chamber, where a burning flame oxidizes the constituents of the fumes. One disadvantage with the foregoing, conventional pyrolysis process is that transferring heat through the floor of the primary chamber is a relatively slow process. Thus, there is generally a long time period required for raising the temperature in the primary chamber and completing the pyrolysis reaction. This time-consuming process can be costly and inefficient. Another disadvantage with the above-described conventional pyrolysis process is that depending upon the type of waste, it may not be possible to reach the required temperature in the primary chamber even if heat is applied through the floor for a long period of time.

Many attempts have been made to develop high efficiency gasification systems which minimize the problems described above including U.S. Pat. No. 4,344,373 A; U.S. Pat. No. 4,135,885 A; U.S. Pat. No. 4,541,841 A and U.S. Pat. No. 4,865,625 A.

However, a need still exists for a highly efficient movable gasification system in which problems associated with undesired tar/oil formation and catalyst contamination are controlled. The present invention accomplishes these goals, and represents an advance in the art of gasification technology.

### SUMMARY OF THE INVENTION

It is an object of the present invention to provide a mobile gasification system and process for carbon-containing materials in a highly efficient manner.

It is another object of the invention to provide a gasification process which is capable of producing substantial amounts of gaseous products from a wide variety of feedstock materials.

It is another object of the invention to provide a gasification process which is simple in design, and uses inexpensive, readily available components.

The invention provides movable carbon-contained materials to liquids system and process. In some embodiments of the invention, a synthesis gas production unit, a synthetic crude production unit and a product cleaning unit are located on a movable platform wherein the units are operationally connected to each other.

Although the invention is illustrated and described herein as embodied in a method for converting carbon-containing raw material into liquids, it is nevertheless not intended to be limited to the details shown, since various modifications may be made therein without departing from the spirit of the invention and within the scope and range of equivalents of the claims.

### BRIEF DESCRIPTION OF THE DRAWINGS

FIG. 1 The principal scheme for the processing unit

FIG. 2 The system in transportation mode

FIG. 3 Functional parts of the system

FIG. 4 The pyrolysis and syngas cleaning units

FIG. 5 The medical and biohazardous wastes preparation unit

FIG. 6 Schematic for a unit to sterilize the medical and biohazardous wastes

FIG. 7 Schematic process flow

FIG. 8 Mobile wastes processing plant

FIG. 9 Extended mobile wastes processing plant

### DETAILED DESCRIPTION OF THE INVENTION

Unless defined otherwise, all technical and scientific terms used herein have the same meaning as commonly understood by one of ordinary skill in the art to which this invention belongs. For the purposes of the present invention, the term syngas (or synthesis gas) refers to the product of a gasification process, and may include carbon monoxide, hydrogen, and carbon dioxide, in addition to other gaseous components such as methane and water.

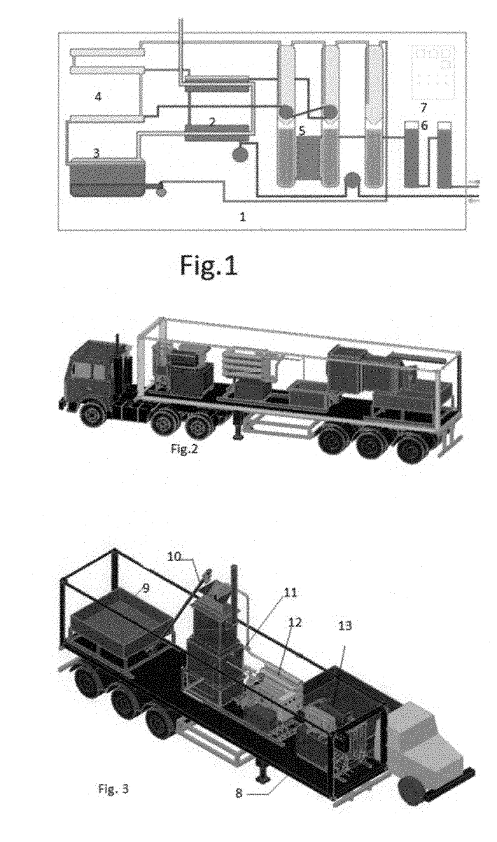

In the Figures, FIG. 1 shows the principal scheme for the mobile processing unit for converting carbon-contained materials to a synthetic hydrocarbon liquid. The mobile processing unit is adapted for treatment of a wide variety of carbon-contained materials including, without limitation, municipal wastes, carbonate, mycelium, coal (including low grade, high sulfur coal not suitable for use in coal-fired power generators), old tires, paper sludge, petroleum coke, heavy oils, biomass, sewage sludge and agricultural wastes. Waste materials suitable for gasification include both hazardous and non-hazardous wastes, such as municipal waste, and wastes produced by industrial activity and biomedical wastes. Examples of biomass useful for gasification include, but are not limited to, waste or fresh wood, remains from fruit, vegetable and grain processing, paper mill residues, straw, grass, and manure. The mobile processing unit is also adapted for processing of biohazardous and medical wastes. The mobile processing unit is comprised of: the standard 40′ container 1; a pre-conditioning unit 2 for initial treatment (or processing) of the carbon-contained materials; cracking reactor with the burner 3; d Diesel fuel stabilization unit 4; distillation columns 5; diesel fuel purification unit 6 and the control panel 7.

As shown in FIG. 1, the mobile processing unit is fully enclosed behind a plurality of panels secured to a frame (not shown), and is built upon a wheeled trailer bed to allow for connection of the processing unit to a truck (not shown) or other similar device for remote transportation to a working site.

FIG. 2 shows the mobile processing unit in transportation mode.

FIG. 3 the the mobile processing unit includes a plurality of main processing components that are secured to a frame 8 and will be described in detail herein below, these include an inlet hopper for receipt of the carbon-contained materials (not shown); compartment 9 for a pre-conditioning and/or initial treatment (or processing) of the carbon-contained materials; a feed-through housing 10 that receives the carbon-contained materials from the grinder (not shown) and through which the carbon-contained material is transferred to a pyrolysis system 11; pyrolysis system 11 converts carbon-contained material into a synthetic gas using known processes and through gas pipeline transferred to gas cleaning and purification system 12 and finally through gas pipeline into a Fischer-Tropsch reactor combined with the product upgrading and recovery system 13. Within the Fischer-Tropsch reactor synthesis gas is being converted into synthetic crude using known processes. Referring to FIG. 3 embodiment of the invention further includes product upgrading and recovery facilities for the conversion of synthetic crude into one or more products, such as naphtha and transportation fuels, including diesel fuel. As used herein, the term “product upgrading” means the refining of a synthetic crude that is waxy, into one or more hydrocarbon products, including for example, a single wide-boiling range product (e.g., C5 to C40) having a reduced pour point which is lower than the waxy synthetic crude which is sufficient to prevent wax crystallization during shipment either as a separate product or blended with crude oil and/or condensate, naphthas, liquefied petroleum gases, base stocks, solvents, kerosene, and hydrocarbon products meeting fuel specifications.

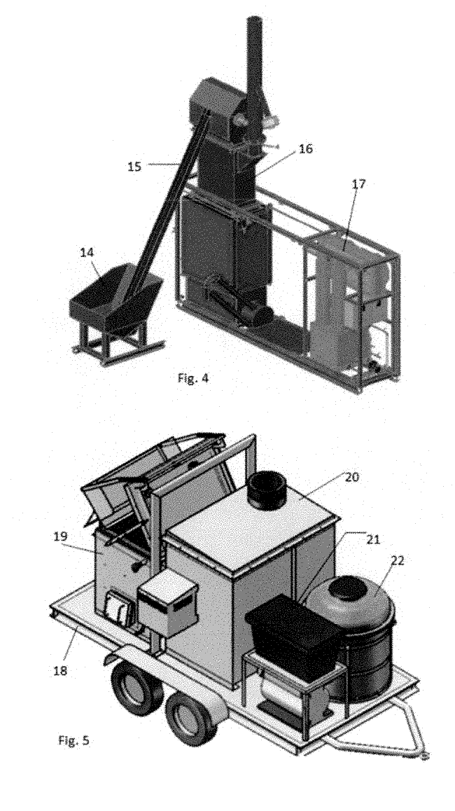

FIG. 4 shows the pyrolysis and gasification unit 16 of the present invention integrated through the pipelines with the gas cleaning system 17. Pyrolysis and gasification units are well known in the arts. Further to FIG. 4 carbon-contained materials from a hopper 14 are transferred into the pyrolysis and gasification unit 16 of the present invention by a transportation conveyer 15 which is also well known in the arts. Further referring to FIG. 4 waste material is described by its ultimate analysis as (CHₓO_{y})¹⁰ and the global gasification reaction may be written as follows:

CHₓO_{y}+wH₂O+m0₂+3.76mN₂->aH₂+bCO+CCO₂+dH₂O+eCH₄+fN,+gC

where w is the amount of water per mole of waste material, m is the amount of O2 per mole of waste, a, b, c, d, e, f and g are the coefficients of the gaseous products and soot (all stoichiometric coefficients in moles). The detailed main reactions are as follows:

CH₄+H₂O->CO+3H₂ (CH4 decomposition-endothermic)

CO+H₂O->CO₂+H₂ (water gas shift reaction-exothermic)

C+H₂O->CO+H₂ (Heterogeneous water gas shift reaction-endothermic)

C+CO₂->2CO (Boudouard equilibrium-endothermic)

2C+->CO₂

A raw synthesis gas product may be characterized by a dirty mixture of gases and solids, comprised of carbon monoxide, hydrogen, carbon dioxide, methane, ethylene, ethane, acetylene, and a mixture of unreacted carbon and ash, commonly called ‘char’, as well as elutriated bed material particulates, and other trace contaminants, including but not limited to ammonia, hydrogen chloride, hydrogen cyanide, hydrogen sulfide, carbonyl sulfide, and trace metals. Syngas may also contain a variety of volatile organic compounds (VOC) or aromatics including benzene, toluene, phenol, styrene, xylene, and cresol, as well as semi-volatile organic compounds (SVOC) or polyaromatics, such as indene, indan, napthalene, methylnapthalene, acenapthylene, acenapthalene, anthracene, phenanthrene, (methyl-) anthracenes/phenanthrenes, pyrene/fluoranthene, methylpyrenes/benzofluorenes, chrysene, benz[a] anthracene, methylchrysenes, methylbenz[a]anthracenes, perylene, ben-zo[a]pyrene, dibenz[a,kl] anthracene, and dibenz[a,h]anthracene.

There is a number of patents relating to different technologies for the synthesis gas cleaning including US 20110126460 A1 and WO 2015089554 A1. The present invention routes the dirty exhaust from the pyrolysis and gasifier 16 into a gas cleaning system 17. Blowers or fans, pumps or other equipment (not shown) can be added to ensure proper flow of the dirty exhaust is maintained. Dry scrubbers with sorbent injectors that introduce limestone or hydrated lime into the gas streams will be added to control any trace of sulfur and nitrogen oxides. Synthesis gas cleanup equipment will consist of packed bed wet scrubbers with sodium hydroxide solutions, absorber vessels, and filters. Small dry scrubbers and/or filters will be used for particulate matter control, while the packed bed scrubbers will be used to neutralize HCl. In addition to the packed bed wet scrubbers, an absorber vessel used for gas purification will be added in to control H2S and COS.

FIG. 5 shows the mobile medical and biohazardous wastes preparation unit. The unit includes a plurality of main processing components that are secured to a frame 18 and will be described in detail herein below; these include an inlet hopper 19 for receipt of materials, a pressure container which fully enclosed behind a plurality of panels 20 and secured to a frame 18 having at least one opening portion with an air-tightly closing means, a means of crushing medical waste, a means of supplying steam 22, a means of exhausting air out of the pressure container and a means of sterilizing the exhausted air 21. Referring to FIG. 5 embodiment of the invention further includes the means of crushing medical waste and the means of supplying steam being positioned within the pressure container.

FIG. 6 shows a schematic view of embodiment for processing medical and biohazardous wastes, provided by the present invention. Further to FIG. 6 the high-pressure steam is required to have a temperature between 150° C. and 170° C. When the temperature is lower than 150° C., the sterilization treatment takes an impractically long time to obtain a sufficient sterilization effect. Further, when it is higher than 170° C., some of waste medical instruments start to decompose, and secondary contamination may occur. When the high-pressure steam has a temperature in the above range, it is preferred to keep the container filled with the high-pressure steam for at least 15 minutes to obtain a sufficient sterilization effect. Further to FIG. 6 waste is fed to a container 23 through an inlet 24 which is one opening of the container 23 formed so as to endure a high-pressure steam. The container 23 has an outlet 25 as another opening. These two openings are structured to be air-tightly closed for the introduction of a high-pressure steam into the container. The fed medical waste passes along a hopper 26 positioned within the container 23 and it is stacked on rotary blades 27 for crushing by biaxial rotary shearing. Then, the inlet 24 is closed to prevent the spread of the medical waste out of the container 23, and the rotary blades 27 are rotated with a motor (not shown) positioned outside the container 23, whereby the medical waste is crushed, and the resultant pieces 28 are stacked on a receiving pan 29 on a receiving bed 30. A valve 31 is a required to open or close the tubing as needed. The sucked air is released into the atmosphere through a sterilization filter 32 positioned on the tubing. Water 34, which is introduced into the container 23 from an external water source (not shown) and whose level is controlled with a water controller 33, is heated with heater 35 to fill the container 1 with a high-pressure steam. The temperature inside the container 1 is controlled with a temperature controller 36.

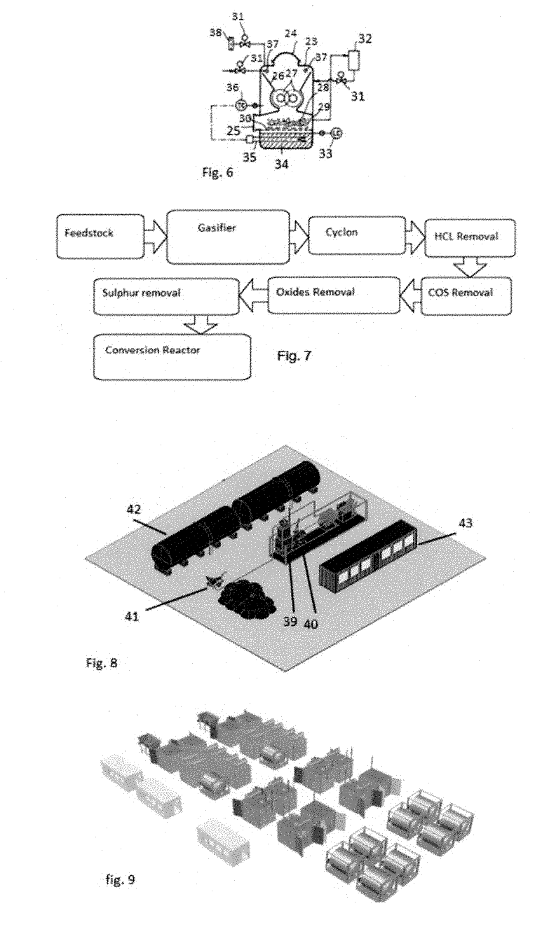

FIG. 7 shows process from the gasification of feedstock stage, through the syngas cleanup stages and the Fischer Tropsch synthesis process for the conversion of clean syngas into ultra clean synthetic fuels.

FIG. 8 shows a schematic view of embodiment for mobile wastes processing plant layout. Further to FIG. 8 the mobile wastes processing plant is comprised of: the standard 40′ container 39; the said mobile processing unit 40; wastes pre-processing unit 41; storage means 42 for produced synthetic hydrocarbon liquids and mobile office 43. Further to FIG. 8 the mobile wastes processing plant of the present embodiment requires industrial ground of 30 meters long and 30 meters wide.

FIG. 9 shows a schematic view of embodiment for extended mobile wastes processing plant layout. Further to FIG. 9 single mobile processing units are connected to form a production chain to increase the processing volume of the carbon-contained materials.

### REFERENCES

1. U.S. Energy Information Administration. (2013, 25 Jul.). International energy outlook 2013: World energy demand and economic outlook, ww.eia.gov/forecasts/ieo/world.cfm
2. Canada-wide Approach for the Management of Wastewater Bio solids. Canadian Council of Ministers of the Environment, 2012
3. Hedden, K., et al., “A New Concept for the Production of Liquid Hydrocarbons from Natural Gas in Remote Areas”, Oil Gas—European Magazine, March 1994, pp. 42-44.
4. Chris Higman and Maarten van der Burgt. Gasification, Second Edition, Elsevier (2008).
5. Beychok, M. R., Process and environmental technology for producing SNG and liquid fuels, U.S. EPA report EPA-660/2-75-011, May 1975
6. Thanapal S S, Annamalai K, Sweeten J, Gordillo G, (2011), “Fixed bed gasification of dairy biomass with enriched air mixture”. Appl Energy, doi:10.1016/j.apenergy.2011.11.072
7. High temperature electrolyte supported Ni-GDC/YSZ/LSM SOFC operation on two-stage Viking gasifier product gas, Ph. Hofmann et al. in Journal of Power Sources 173 (2007) 357-366
8. Fundamental Issues in Control of Carbon Gasification Reactivity edited by L. Lahaye, Pierre Ehrburger Centre de Recherches sur la Physico-Chimie des Surfaces Solides—C.N.R.S., Mulhouse, France, 2012
9. Encyclopedia of Electrochemical Power Sources edited by Chris K. Dyer, Patrick T. Moseley, Zempachi Ogumi, David A. J. Rand, Bruno Scrosati. Elsevier, 2009
10. A. Mountouris, E. Voutsas, D. Tassios, 2006 Solid waste plasma gasification: Equilibrium model development and exergy analysis. Energy Conyers. Manage. 4717231737
11. U.S. Environmental Protection Agency (2010 Municipal solid waste in the United States: 2009 Facts and Figures Washington, D.C.
12. A. S. An′shakov, V. A. Faleev, A. A. Danilenko, E. K. Urbakh, A. E. Urbakh, 2007 Investigation of plasma gasification of carbonaceous technogeneous wastes. Thermophys. Aeromech. 14607616

### Patent Citations

| Cited Patent | Filing date | Publication date | Applicant | Title |
|---|---|---|---|---|
| U.S. Pat. No. 2,527,197 A | 17 Feb. 1945 | 24 Oct. 1950 | Walter F Rollman | Method of producing a |
| | | | | carbon monoxide and |
| | | | | hydrogen gas mixture from |
| | | | | carbonaceous materials |
| U.S. Pat. No. 2,879,148 A | 6 Dec. 1955 | 24 Mar. 1959 | Harold V Atwell | Process for the production of |
| | | | | carbon monoxide from a solid |
| | | | | fuel |
| U.S. Pat. No. 3,009,795 A | 10 Nov. 1958 | 21 Nov. 1961 | Harold V Atwell | Gasification of solid |
| | | | | carbonaceous materials |
| U.S. Pat. No. 2,618,544 A | 29 Jun. 1951 | 18 Nov. 1952 | Herbert G M | Gasification of carbonaceous |
| | | | Fischer, Jr Albert B | materials |
| | | | Welty | |
| U.S. Pat. No. 3,544,291 A | 22 Apr. 1968 | 1 Dec. 1970 | Warren G | Coal gasification process |
| | | | Schlinger, William L | |
| | | | Slater | |
| U.S. Pat. No. 3,904,386 A | 26 Oct. 1973 | 9 Sep. 1975 | Ernest E Donath, | Combined shift and |
| | | | Michael S | methanation reaction process |
| | | | Graboski | for the gasification of |
| | | | | carbonaceous materials |
| U.S. Pat. No. 4,265,868 A | 8 Feb. 1978 | 5 May 1981 | John F. Kamody | Production of carbon |
| | | | | monoxide by the gasification |
| | | | | of carbonaceous materials |
| U.S. Pat. No. 4,642,125 A | 30 Nov. 1983 | 10 Feb. 1987 | Maksymilian | Carbonaceous material and |
| | | | Burk, Jack L. | methods for making |
| | | | Blumenthal | hydrogen and light |
| | | | | hydrocarbons from such |
| | | | | materials |
| U.S. Pat. No. 3,976,442 A | 18 Dec. 1974 | 24 Aug. 1976 | Peter L. Paull, | Synthesis gas from gaseous |
| | | | Warren G. | CO2-solid carbonaceous fuel |
| | | | Schlinger | feeds |
| U.S. Pat. No. 5,362,443 A | 27 Dec. 1991 | 8 Nov. 1994 | Mitsuyuki Tanaka | Method and apparatus for |
| | | | | disposal of medical waste |
| WO2011129878 A2 | 11 Apr. 2011 | 20 Oct. 2011 | Bhagya Chandra | Methods for gasification of |
| | | | Sutradhar, Ching- | carbonaceous materials |
| | | | Whan Ko | |
| U.S. Pat. No. 5,961,673 A | 26 Jun. 1996 | 5 Oct. 1999 | Norbert Topf, | Process and apparatus for |
| | | | Guenter Reichert | producing fuel gas |
| U.S. Pat. No. 6,170,770 B1 | 11 Aug. 1999 | 9 Jan. 2001 | John W. Rich, Jr. | Process and apparatus for |
| | | | | preparing feedstock for a coal |
| | | | | gasification plant |
| U.S. Pat. No. 4,039,425 A | 22 Dec. 1975 | 2 Aug. 1977 | Richard C. Neavel | Method for preparing a coal |
| | | | | slurry substantially depleted |
| | | | | in mineral-rich particles |
| U.S. Pat. No. 4,061,274 A | 26 Jul. 1976 | 6 Dec. 1977 | Robert M. Williams | Material reducing apparatus |
| | | | | and method of operating the |
| | | | | same |
| U.S. Pat. No. 3,826,208 A | 6 Aug. 1973 | 30 Jul. 1974 | Williams R | Apparatus and system for |
| | | | | disposing of combustible and |
| | | | | waste material |
| US20090007484 A1 | 15 Sep. 2008 | 8 Jan. 2009 | David G. Smith | Apparatus and process for |
| | | | | converting biomass feed |
| | | | | materials into reusable |
| | | | | carbonaceous and |
| | | | | hydrocarbon products |
| CA2185330 A1 | 10 Mar. 1995 | 14 Sep. 1995 | Bruno Aubert, | Method for sterilising |
| | | | Serge Hauss | hazardous waste, particularly |
| | | | | hospital waste, using high |
| | | | | frequency treatment, and |
| | | | | device therefor |
| U.S. Pat. No. 7,354,550 B2 | 6 Sep. 2001 | 8 Apr. 2008 | Bruno Aubert | Disinfection or sterilisation |
| | | | | method |
| US20140251923 A1 | 19 May 2014 | 11 Sep. 2014 | Mauri Salmisuo, | Solids separator and method |
| | | | Juha Mattila, Teppo | of treatment for bio-waste |
| | | | Nurminen | |
| U.S. Pat. No. 4,342,830 | 11 May 1981 | 3 Aug. 1982 | Clifford C. Holloway | Process for separating and |
| | | | | recovering organics and |
| | | | | inorganics from waste |
| | | | | material |
| U.S. Pat. No. 5,941,468 | 22 Jan. 1998 | 24 Aug. 1999 | Robert W. Lewis, | Waste treatment apparatus |
| | | | Randall G. McKee | and method |
| U.S. Pat No. 5,333,146 A | 27 Jul. 1992 | 26 Jul. 1994 | Murry Vance | On-site, biohazardous waste |
| | | | | disposal system |
| U.S. Pat No. 8,123,731 B2 | 21 Aug. 2002 | 28 Feb. 2012 | Marshall C. Ryan | Method and apparatus for |
| | | | | disposing of liquid surgical |
| | | | | waste for protection of |
| | | | | healthcare workers |
| U.S. Pat. No. 6,348,174 B1 | 6 Dec. 1999 | 19 Feb. 2002 | John L. Hall | Biohazardous waste source |
| | | | | sterilizer system and |
| | | | | processing method |
| U.S. Pat. No. 6,360,679 | 7 Oct. 1999 | 26 Mar. 2002 | Howard Morgan | Sanitary waste disposal unit |
| | | | Clarke | |
| U.S. Pat. No. 5,084,250 A | 29 Jan. 1990 | 28 Jan. 1992 | John Hall | Apparatus for treating and |
| | | | | disposing of bio-hazardous |
| | | | | waste and solid waste |
| U.S. Pat. No. 6,867,393 B1 | 5 Dec. 2003 | 15 Mar. 2005 | Robert W. Lewis | Steam sterilization system for |
| | | | | sterilizing medical waste |
| U.S. Pat. No. 4,344,373 A | 30 Oct. 1979 | 30 Aug. 1982 | Yoshiaki Ishii | Method for pyrolyzing |
| U.S. Pat. No. 4,135,885 A | 3 Jan. 1977 | 23 Jan. 1979 | Alex F. Wormser | Burning and desulfurizing coal |
| U.S. Pat. No. 4,541,841 A | 16 Jun. 1982 | 17 Sep. 1985 | Dieter Reinhardt | Method for converting |
| | | | | carbon-containing raw |
| | | | | material into a combustible |
| | | | | product gas |
| U.S. Pat. No. 4,865,625 A | 2 May 1988 | 12 Sep. 1989 | Lyle K. Mudge | Method of producing |
| | | | | pyrolysis gases from carbon- |
| | | | | containing materials |
| WO 2015089554 A1 | 16 Dec. 2013 | 25 Jun. 2015 | Chun-zhu Ll, | Process and apparatus for |
| | | | Richard | cleaning raw product gas |
| | | | GUNAWAN, Li | |
| | | | Dong | |
| US 20110126460 A1 | 26 May 2009 | 2 Jun. 2011 | Leslie Bromberg | Regenerator for syngas |
| | | | | cleanup and energy recovery |
| | | | | in gasifier systems |

1. A carbon-containing materials to liquids process comprising the steps of:
a. transporting a movable platform comprising a material preparation unit, synthesis gas production unit, a synthetic crude production unit, and a product clean up unit to a location at or near carbon-containing materials containing reserve;
b. receiving carbon-containing materials from the reserve and biohazard wastes from the appropriate source;
c. shredding said waste, and transporting said shredded waste into a neutralization chamber;
d. neutralizing biohazardous waste with the super-heated steam and transporting neutralized said wastes into a mixing chamber;
e. mixing said neutralized wastes and carbon-containing materials;
f. converting the said mix into a synthesis gas in the synthesis gas production unit;
g. converting synthesis gas produced in step f by a Fischer Tropsch (F-T) process into a finished end-user product.

2. The process of claim 1 wherein use of water gas shift with, and without electrolysis in the syngas stream to attain gas equilibrium, and enrich syngas with additional hydrogen.

3. The process of claim 1 wherein the synthesis gas produced in step f has an H₂/CO ratio equal to or greater than 2.

4. The process of claim 1 further including the step of increasing the H₂/CO ratio of the synthesis gas produced by the gasification of the said mix by the water-gas shift reaction.

5. The process of claim 1 further including the step of converting synthesis gas produced by steps f to hydrocarbon products by Fischer-Tropsch synthesis.

6. The process of claim 1 wherein the feed to the said mix liquefaction of step g includes hydrogen from the synthesis gas produced by steps f.

7. The process of claim 1 wherein the movable platform further comprises a utilities unit operationally connected to the system, wherein the utilities system comprises a cooling water system and industrial electrical power connection.

8. The process of claim 1 wherein the neutralizing biohazardous waste said in step c comprising carrying out the steps of:
a. shearing biohazardous waste in a closed container having an internal wall with a device to expose an interior portion of the biohazardous waste to direct contact with a high-pressure steam;
b. generating and filling the container with the high-pressure steam having a temperature between 150° C. and 170° C. to sterilize the sheared biohazardous waste, the internal wall of the container and the device to expose the interior portion of the biohazardous waste within the container.

9. A process according to claim 1, wherein the sheared biohazardous waste is sterilized by the high-pressure steam for at least 20 minutes.

10. A process according to claim 1, wherein the other device is a means for crushing biohazardous waste.

11. A process according to claim 1, wherein the air is sterilized by at least one means selected from the group consisting of filtering means, heating means and chemical oxidation means.

12. A system for disposing of biohazardous waste having non-exposed interior portions, comprising a pressure container having at least one top opening portion with a means for closing said at least one top opening providing for an airtight closing, a means for crushing biohazardous waste which is capable of shearing the biohazardous waste to expose the interior portions of the biohazardous waste to direct contact with high-pressure steam, a means for generating steam below said crushing means, said steam generating means comprising water filled in a lower portion of the pressure container and a heater provided within the water, a means for exhausting air out of the pressure container and a means for sterilizing the exhausted air, wherein the means for crushing biohazardous waste and the means for generating steam are positioned within the pressure container.

13. A system according to claim 11, wherein the pressure container has an opening formed in an upper portion of the pressure container which is an inlet for feeding biohazardous waste therein and another opening formed in a lower portion of the pressure container which is an outlet for discharging the sheared and sterilized biohazardous waste.

14. A system according to claim 11, wherein the pressure container has a movable receiving pan for receiving sheared biohazardous waste, the receiving pan being positioned above the water and having a plurality of pores at least in a bottom thereof.

15. A system according to claim 11, wherein the means for exhausting air comprises a pipe communicating with the pressure container and an air-sucking pump connected to the pipe.

16. A system according to claim 11, wherein the means for sterilizing the exhausted air is at least one means selected from the group consisting of a filtering means, a heating means and a chemical oxidation means, positioned in a pipe connecting the pressure container and a sucking pump.

17. A system according to claim 11, wherein the pressure container further has a means for jetting water to cool the steam.

18. A method of gasifying a carbon-containing material said in step e, comprising of:
1. reacting the carbon-containing material with steam in presence of a catalyst thus producing a gas product including CO, CO₂, CH₄, H₂O and H₂;
2. thermally decomposing CH₄ generated in stage 1 into C and H₂; and
3. converting CO₂ generated in stage 1 into CO using the product of stages 2 and 3

19. The method of claim 17, further comprising recirculating at least a part of carbon generated in 2 to 3 of claim 17 which gasifies the carbon-containing material.

20. The method of claim 17, further comprising recirculating H₂ and CO generated in 2 to 3 of claim 17.

21. The method of claim 18, further comprising recirculating H₂ and CO generated in 1 to 3 of claim 17.

22. The method of claim 17, wherein 3 is carried out using any one selected from among a reverse water-gas shift reaction, a hydrogenation reaction, a CO₂ reforming reaction, and a C—CO₂ gasification reaction.

23. The method of claim 17, wherein the carbon-containing material is coal, low quality brown coal, coal chips and dust, biomass, waste, heavy oil, or petroleum coke, crude oil, used tires, municipal and biohazardous wastes.

24. The method of claim 17 yields 1-1.5 m³ of said synthesis gas per 1 kilogram of low quality brown coal and municipal wastes.

25. The process of claim 1 wherein the said synthesis gas reacting in the presence of a hydrocarbon synthesis catalyst to produce heavier hydrocarbons.
